(19) United States
(12) Patent Application Publication
(10) Pub. No.: US 2010/0204444 A1
(43) Pub. Date: Aug. 12, 2010

(54) MULTIFUNCTIONAL PROTEIN SIMULTANEOUSLY DELIVERING ANTIBODIES AND NANOPARTICLES

(75) Inventors: Bong Hyun Chung, Daejeon (KR); Yong Taik Lim, Daejeon (KR); Mi Young Cho, Daejeon (KR); Jeong Min Lee, Daejeon (KR)

Correspondence Address: MOORE & VAN ALLEN PLLC, P.O. BOX 13706, Research Triangle Park, NC, 27709, US

(73) Assignee: KOREA RESEARCH INSTITUTE OF BIOSCIENCE AND BIOTECHNOLOGY, Daejeon (KR)

(21) Appl. No.: 12/670,245

(22) PCT Filed: Jul. 25, 2008
(86) PCT No.: PCT/KR08/04376

§ 371 Date: Mar. 16, 2010

(30) Foreign Application Priority Data
Jul. 25, 2007 (KR) 10-2007-0074759

(51) Int. Cl. C07K 16/00; C07K 2/00; C12N 9/96; C07K 7/06; C07H 21/00; C12N 15/63; C12P 21/06; C12N 1/00

(52) U.S. Cl. 530/322; 530/300; 435/188; 530/350; 530/327; 536/23.1; 435/320.1; 435/69.1; 530/391.1; 435/243

(57) ABSTRACT

The present invention relates to a polypeptide structure which can simultaneously deliver an antibody and a nanoparticle into cells, more specifically, to a polypeptide structure for intracellular delivery of an antibody and a nanoparticle, which comprises a nanoparticle-binding region, an antibody-binding region and a signaling capable of delivering substances into cells.

### TECHNICAL FIELD

The present invention relates to a polypeptide structure which can simultaneously deliver an antibody and a nanoparticle into cells, and more particularly to a polypeptide structure for intracellular delivery of an antibody and a nanoparticle, which comprises a nanoparticle-binding region, an antibody-binding region and a signaling molecule capable of delivering substances into cells.

### BACKGROUND ART

Metal nanoparticles having the properties of strongly absorbing or scattering light, quantum dot nanoparticles having excellent fluorescent properties and light stability, and magnetic particles, which can be used for separation of a specific organelle or as MR imaging contrast agents, are typical nanoparticles which are expected to be used in various applications in the biological and medical fields due to the unique properties thereof. Such nanoparticles image cells and tissues by binding to antibodies targeting specific portions of cell membrane surfaces. Recently, studies focused on by intracellular delivery properties of peptides attaching peptides having intracellular delivery properties to nanoparticle surfaces have been actively conducted (Nitin, N. et al., J. Biological Inorganic Chemistry, 9:706, 2004, Mahesh, D. et al., J. Nanoscience and Nanotechnology, 6:2651, 2006, Mackay, J. A. et al., J. Dispersion Science and Technology, 24:465, 2003).

In order to simultaneously immobilize peptides and antibodies, which have different functions, to nanoparticles, the surface properties of the nanoparticles must be controlled such that the peptides and the antibodies can be simultaneously attached to the nanoparticles. In addition, if peptides and antibodies are simultaneously attached to nanoparticles, there is problem in that the relative amounts of the peptides and the antibodies to be attached to the nanoparticle surfaces, cannot be systemically controlled.

Thus, there is an urgent need to develop multifunctional biomolecules, which enable antibodies and nanoparticles, having various functions, to be delivered into specific cells regardless of the kinds thereof.

Accordingly, the present inventors have prepared a polypeptide structure by introducing functional groups, such as histidine, GST, MBP and the like, capable of binding to nanoparticles, into one end of protein G having the property of binding to the specific region of antibodies, and attaching specific peptides (cell penetration peptides), having intracellular delivery properties, to the other end of the protein, and have found that the polypeptide structure can simultaneously deliver antibodies and functional nanoparticles into cells, thereby completing the present invention.

### SUMMARY OF INVENTION

It is an object of the present invention to provide a polypeptide structure having the function of simultaneously delivering an antibody and a functional nanoparticle into cells.

Another object of the present invention is to provide a multifunctional complex, in which an antibody and a nanoparticle are bound to said polypeptide structure.

To achieve the above objects, in one aspect, the present invention provides a polypeptide structure for intracellular delivery of an antibody and a nanoparticle, which comprises a nanoparticle-binding region, an antibody-binding region and a signaling molecule capable of delivering substances into cells.

In another aspect, the present invention provides a DNA encoding said polypeptide structure, a recombinant vector containing said DNA, a recombinant microorganism transformed with said recombinant vector, and a method for preparing said polypeptide structure, which comprises culturing said recombinant microorganism.

In still another aspect, the present invention provides a multifunctional complex, in which an antibody and a nanoparticle are bound to said polypeptide structure.

Other features and aspects of the present invention will be apparent from the following detailed description and the appended claims.

### BRIEF DESCRIPTION OF DRAWINGS

FIG. 1 is a schematic diagram of the inventive multifunctional protein structure, which can simultaneously deliver antibodies and nanoparticles into cells.

FIG. 2 is a schematic diagram showing targeting a specific organelle in cells using a multifunctional protein according to the present invention.

FIG. 3 shows the structures of genes and vectors, which are used to prepare functional proteins according to the present invention.

FIG. 4 shows the results of electrophoresis conducted after the expression and purification of multifunctional proteins designed in FIG. 3.

FIG. 5 shows the intracellular delivery properties of multifunctional proteins prepared according to the present invention.

FIG. 6 shows the results of mitochondria targeting performed using multifunctional proteins according to the present invention and magnetic nanoparticles.

FIG. 7 shows the results of Western blot analysis of mitochondria separated using the technique of FIG. 6.

### DETAILED DESCRIPTION OF THE INVENTION AND PREFERRED EMBODIMENTS

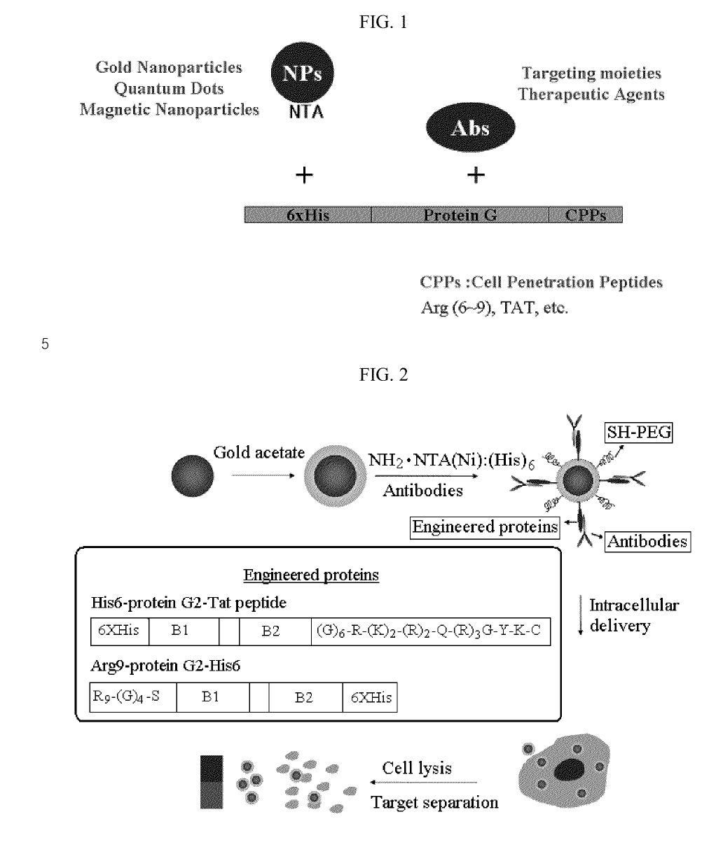

In the present invention, a recombinant polypeptide structure, which has a portion having the property capable of binding selectively to the Fc domain of antibodies, a portion having reactive groups, capable of binding to nanoparticles, at both ends thereof, and a portion comprising signaling molecules capable of passing through a cellular or nuclear membrane, was prepared (FIG. 1).

Also, after magnetic nanoparticles were delivered into cells using this polypeptide structure, it was shown that the magnetic nanoparticles could selectively target and separate mitochondria (FIG. 2).

Accordingly, in one aspect, the present invention relates to a polypeptide structure for intracellular delivery of an antibody and a nanoparticle, which comprises a nanoparticle-binding region, an antibody-binding region and a signaling molecule capable of delivering substances into cells.

In the present invention, the antibody-binding region is preferably selected from the group consisting of protein G, protein A, a protein A/G mixture, protein L, antibody-binding peptides and antibody-binding nucleotides, which can bind selectively to the Fc-domain of antibodies.

In the present invention, the nanoparticle-binding region is preferably selected from the group consisting of poly-histidine, glutathione S-transferase (GST), maltose-binding protein (MBP), myc, hemagglutinin (HA), cystein, streptavidin, polyarginine, elastin (ELP)-based biopolymer, a galactose-binding domain, a calmodulin-binding domain, a chitin-binding domain, a cellulose-binding domain, a thioredoxine-binding domain, an intein binding domain, a S-peptide-binding domain, and a DNA.

In the present invention, the signaling molecules capable of passing through the cellular or nuclear membrane are preferably cell penetration signaling peptides. The cell penetration signaling peptides include oligoarginine, a TAT-peptide (YGRKKRRQRRR), a drosophila-derived Antp peptide, a VP22 peptide, mph-1-btm (USP 2005/0147971), and cell penetration signaling peptides found using various phage display techniques.

In the present invention, the nanoparticles are preferably selected from the group consisting of light-absorbing/scattering nanoparticles, fluorescent nanoparticles and magnetic nanoparticles. The light-absorbing/scattering nanoparticles are preferably Au or Ag nanoparticles, the fluorescent nanoparticles are preferably nanoparticles of a material selected from the group consisting of CdSe, CdSe/ZnS, CdTe/CdS, CdTe/CdTe, ZnSe/ZnS, ZnTe/ZnSe, PbSe, PbS InAs, InP, InGaP, InGaP/ZnS and HgTe, or nanoparticles in which an organic or inorganic fluorescent dye is dispersed in a material selected from the group consisting of silica, titanium or polymers, and the magnetic nanoparticles are preferably nanoparticles of a material selected from the group consisting of Fe₂O₃, Fe₃O₄, FePt, Co and gadolinium (Gd).

In the present invention, the antibodies can selectively target various organelles in the cytoplasm or nucleus. Also, the antibodies are preferably antibodies having therapeutic functions.

The polypeptide structure of the present invention can be prepared in a large amount by transforming a gene, encoding the polypeptide structure, into easy-to-use E. coli, Bacillus, yeasts such as Saccharomyces cerevisiae or Pichia pastoris, or animal cells such as CHO cells, and purifying proteins from the transformed microorganisms through a simple process.

Accordingly, in another aspect, the present invention relates to a DNA encoding the polypeptide structure, a recombinant vector containing said DNA, microorganisms transformed with said recombinant vector, and a method for preparing said polypeptide structure, which comprises culturing said transformed microorganisms.

### EXAMPLES

Hereinafter, the present invention will be described in further detail with reference to examples. It is to be understood, however, that these examples are for illustrative purposes only and are not to be construed to limit the scope of the present invention.

In the following examples, like reference numerals denote like elements, and various elements and regions in the drawings are illustrated schematically. Accordingly, the present invention is not limited by the relative sizes or distances illustrated in the attached drawings.

### Example 1

### Analysis of Protein Expression of Staphylococcal Protein G Variants

In order to construct His6-protein G-TAT peptide, Arg9-protein G-His6, His6-protein G and protein G-His6 genes, which are Staphylococcal protein G varients, 6 primers were constructed in the following manner.

To construct the His6-protein G-TAT peptide gene, 6 histidine codons (CAC) were added to the N-terminus of Staphylococcal protein G after the ATG start codon, and GGC GGC GGC GGC GGC CGT AAA AAA CGT CGT CAG CGT CGT CGT GGC TAT AAA TGC (SEQ ID NO: 1; 4 glycine codons as a linker with protein G and a signal peptide ((G)-R—(K)2-(R)2-Q-(R)3-G-Y—K—C)) codon allowing the protein to enter cells) was added to the C terminus of staphylococcal protein G before the TAA termination codon.

The Arg9-protein G-His6 gene was constructed by adding 9 arginine codons (CGT) to the N-terminus of staphylococcal protein G after the ATG start codon and adding 6 CAC (histidine) codons to the C terminus of staphylococcal protein G before the TAA termination codon.

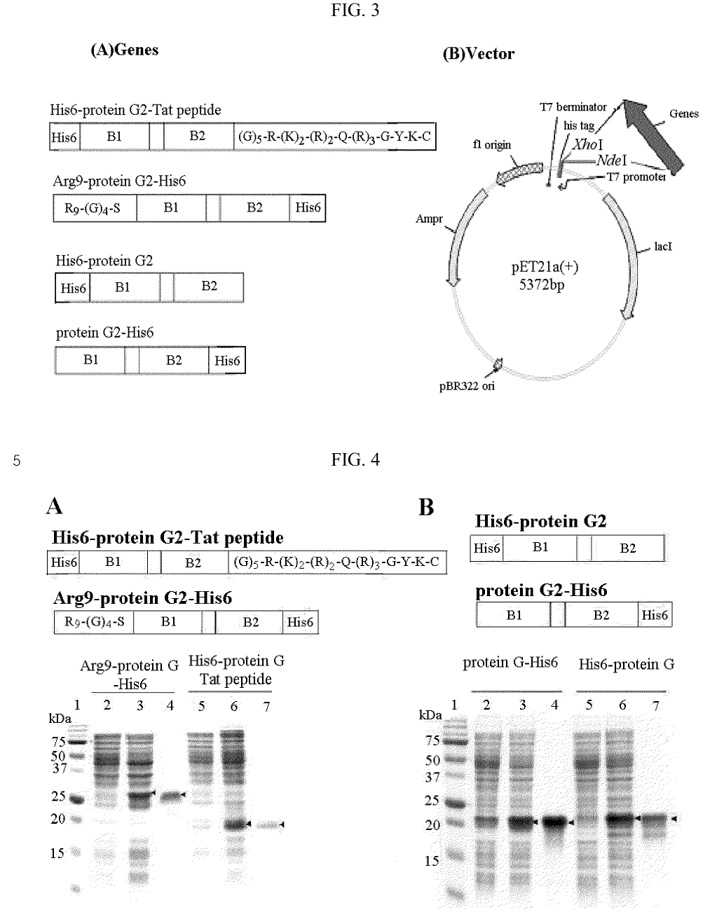

The His6-protein G gene was constructed by adding 6 histidine codons (CAC) to the N terminus of staphylococcal protein G after the ATG start codon, and the protein G-His6 gene was constructed by adding 6 histidine codons (CAC) to the C terminus of staphylococcal protein G before the TAA termination codon (FIG. 3A).

In order to insert the genes into the expression vector pET21a (Novagen), an NdeI restriction enzyme site was introduced into an N-terminal primer, and an XhoI restriction enzyme site was introduced into a C-terminal primer.

Staphylococcus sp. genomic gene (KCCM 41566) of the Streptococus Lancefield's group G strain was subjected to polymerase chain reaction (PCR) with primers (a primer pair of SEQ ID NO: 2 and SEQ ID NO: 7 for His6-protein G; a primer pair of SEQ ID NO: 4 and SEQ ID NO: 6 for protein G-His6; a primer pair of SEQ ID NO: 2 and SEQ ID NO: 5 for His6-protein G-TAT peptide; and a primer pair of SEQ ID NO: 3 and SEQ ID NO: 6 for Arg9-protein G-His6), thus obtaining only amino acid fragments (B1 [cutting form of 10 amino acids at the front part] and B2) known as domains binding to antibodies. The obtained fragments were digested with the restriction enzyme introduced into each of the primers, and then inserted into a pET21a vector, treated with NdeI and XhoI restriction enzymes, thus constructing pET-His6-protein G-TAT peptide, pET-Arg9-protein G-His6, pET-His 6-protein G and pET-protein G-His6 vectors, respectively (FIG. 3B). The expression vectors express Met at the N terminus.

Primer 1 (sense; SEQ ID NO: 2):
5′-CATATGCACCACCACCACCACCACAAAGGCGAAACAACTACTGAAGC
T-3′
Primer 2 (sense; SEQ ID NO: 3):
5′-CATATGCGTCGTCGTCGTCGTCGTCGTCGTCGTGGCGGCGGCGGCAG
CAAAGGCGAAACAACTACTGAAGCT-3′
Primer 3 (sense; SEQ ID NO: 4):
5′-CATATGAAAGGCGAAACAACTACTGAAGCT-3′
Primer 4 (antisense; SEQ ID NO: 5):
5′-CTCGAGTTAGCATTTATAGCCACGACGACGCTGACGACGTTTTTTAC
GGCCGCCGCCGCCGCCTTCAGTTACCGTAAAGGTCTTAGTC-3′
Primer 5 (antisense; SEQ ID NO: 6):
5′-CTCGAGTTCAGTTACCGTAAAGGTCTTAGTC-3′
Primer 6 (antisense; SEQ ID NO: 7):
5′-CTCGAGTTATTCAGTTACCGTAAAGGTCTTAGT-3′

E. coli BL21 cells, transformed with each of the pET-His6-protein G-TAT peptide, pET-Arg9-protein G-His6, pET-His6-protein G and pET-protein G-His6 vectors, was shake-cultured at 37° C. until it reached an OD of 0.6 at 600 nm, and then isopropyl β-D-thiogalactopyranoside (IPTG) was added thereto to a final concentration of 1 mM. Then, the cells were cultured at 25° C. to induce the expression of each of the staphylococcal protein G variants. After 12 hours, the E. coli cells, obtained by centrifugation, were disrupted with ultrasonic waves (Branson, Sonifier 450, 3 KHz, 3 W, 5 min), and then a solution of total protein was collected and separated by centrifugation into a solution of soluble fraction proteins and a solution of non-soluble fraction proteins. Each of the solutions was collected.

Each of the protein solutions mixed with a buffer solution (12 mM Tris-Cl, pH 6.8, 5% glycerol, 2.88 mM mercaptoethanol, 0.4% SDS, 0.02% bromophenol blue), was heated at 100° C. for 5 minutes, and was then loaded onto polyacrylamide gel consisting of 1-mm thick 15% separation gel (pH 8.8, 20 cm in width and 10 cm in length) covered by a 5% stacking gel (pH 6.8, width 10 cm and length 12 cm). Then, each of the loaded solutions was electrophoresed at 200-100 V and 25 mA for 1 hour, and the gel was stained with Coomassie dye to visualize recombinant proteins (FIG. 4).

In FIG. 4A, lane 1 is a protein size marker, lane 2 is the total protein of E. coli transformed with pET-Arg9-protein G-His6, lane 3 is the soluble fraction protein of E. coli transformed with pET-Arg9-protein G-His6, lane 4 is a protein purified and eluted from the soluble fraction protein of E. coli transformed with the plasmid pET-Arg9-protein G-His6, lane 5 is the total protein of E. coli transformed with the pET-His6-protein G-TAT peptide, lane 6 is the soluble fraction protein of E. coli transformed with the plasmid pET-His6-protein G-TAT peptide, and lane 7 is a protein purified and eluted from the soluble fraction protein of E. coli transformed with the plasmid pET-His6-protein G-TAT peptide.

In FIG. 4B, lane 1 is a protein size marker, lane 2 is the total protein of E. coli transformed with pET-His6-protein G, lane 3 is the soluble fraction protein of E. coli transformed with pET-His6-protein G, lane 4 is a protein purified and eluted from the soluble fraction protein of E. coli transformed with pET-His6-protein G, lane 5 is the total protein of E. coli transformed with pET-protein G-His6, lane 6 is the soluble fraction protein of E. coli transformed with pET-protein G-His6, and lane 7 is a protein purified and eluted from the soluble protein fraction of E. coli transformed with pET-protein G-His6.

In order to purify the above-obtained soluble protein solution, a solution of disrupted cells, in which the four recombinant genes conjugated with hexahistidine were expressed, was loaded on a column packed with IDA excellulose. The six recombinant proteins conjugated with histidine were eluted with an eluent (50 mM Tris-Cl, 0.5M imidazole, 0.5M NaCl, pH8.0). The eluted protein solution was dialyzed in PBS buffer (phosphate-buffered saline, pH7.4).

### Example 2

### Delivery of Multifunctional Proteins into Cells

The intracellular delivery properties of the protein G variants (His6-protein G-TAT peptide, Arg9-protein G-His6, His6-protein G, and protein G-His6), prepared in Example 1, were examined. The protein G variants were bound to FITC-labeled antibody, such that they could be observed with a fluorescent microscope. The TAT-peptide used in this Example consisted of YGRKKRRQRRR, and the oligoarginine contained 6-12 arginines.

As a result, as shown in FIG. 5, it could be seen that the protein G variant, in which TAT peptide and oligoarginine, having intracellular delivery properties, were attached to the protein G structure, had excellent intracellular delivery properties. However, it could be seen that the protein G variant containing no cell penetration peptide did not enter cells.

### Example 3

### Delivery of Recombinant Proteins Attached to Nanoparticle Surface and Cell Imaging

His 6-protein G-TAT peptide and Arg9-protein G-His6, confirmed to have intracellular delivery properties on the basis of the results obtained in Example 2, were coated on nanoparticles. Nanoparticles usable in this Example include gold nanoparticles, quantum dot nanoparticles and iron oxide nanoparticles, and in this Example, each of the two recombinant proteins, His6-protein G-TAT peptide and Arg9-protein G-His6, was attached to the surface of iron oxide nanoparticles. The surface of iron oxide nanoparticles prepared in aqueous solution was substituted with NH₂—NTA(Ni), such that the His6 portion of the two recombinant proteins could be attached to the NTA(Ni) portion of the nanoparticles. Also, after the surfaces of gold nanoparticles and quantum dot nanoparticles are substituted with NH₂—NTA(Ni), the recombinant proteins can be attached to the nanoparticles (FIG. 2).

### Example 4

### Mitochondria Targeting and Separation Using Magnetic Nanoparticles

Antibodies [(FITC)-anti-mitochondria] capable of targeting the intracellular organelle, mitochondria were attached to the iron oxide nanoparticle-His6-protein G-TAT peptide and iron oxide nanoparticle-Arg9-protein G-His6 prepared in Example 3. Herein, the antibodies were bound selectively to the protein G portion of the recombinant proteins already attached to the nanoparticle surfaces.

Each of the above-prepared FITC antibody-iron oxide nanoparticle-His6-protein G-TAT peptide and FITC antibody-iron oxide nanoparticle-Arg9-protein G-His6 was incubated with HeLa cells.

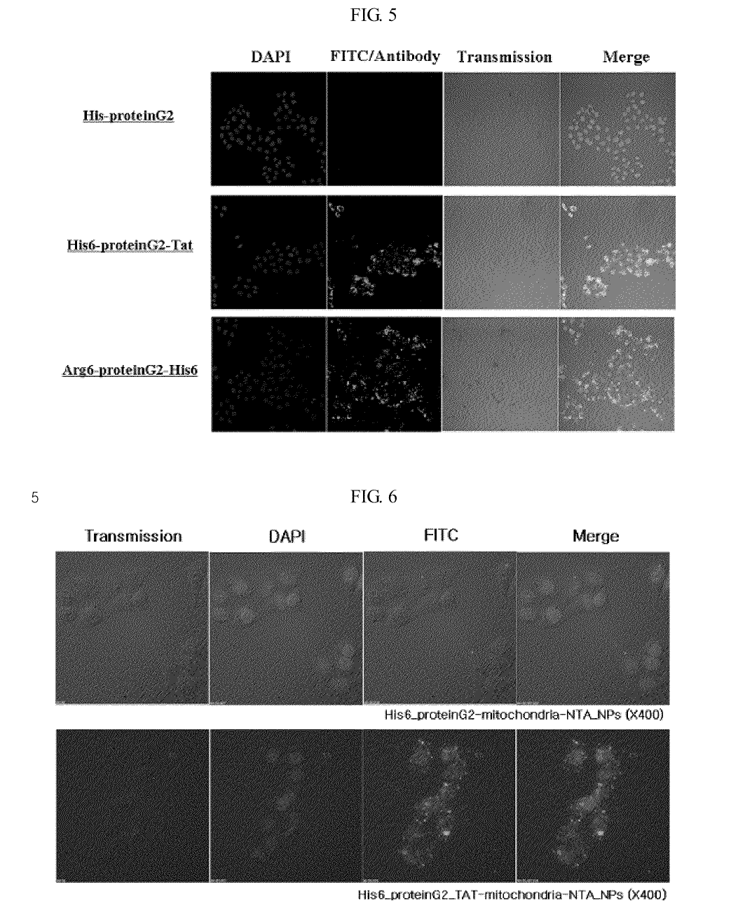

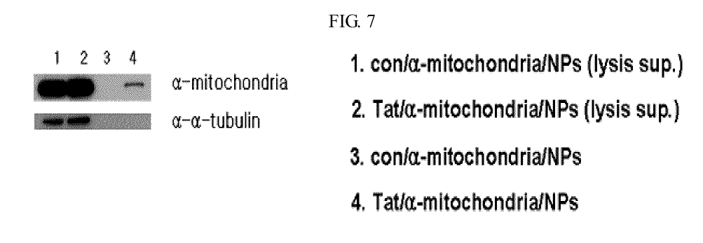

As a result, as shown in FIG. 6, it was observed that the FITC-labeled magnetic nanoparticles were very well delivered into the cells. The targeted magnetic nanoparticles and mitochondria can be selectively recovered using a magnet after cell lysis (FIG. 2). In addition, as shown in FIG. 7, the mitochondria targeted with the magnetic nanoparticles can also be selectively separated through Western blot analysis.

### INDUSTRIAL APPLICABILITY

As described above, the functional polypeptide structure according to the present invention can simultaneously deliver a therapeutic antibody and a nanoparticle having various functions, into cells, and thus are useful for the targeting of specific intracellular organelles, cell imaging, the separation of a specific intracellular organelle, and the like.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A polypeptide structure for intracellular delivery of an antibody and a nanoparticle, which comprises a nanoparticle-binding region, an antibody-binding region and a signaling molecule capable of delivering substances into cells.

2. The polypeptide structure according to claim 1, wherein the nanoparticle-binding region is selected from the group consisting of poly-histidine, glutathione S-transferase (GST), maltose-binding protein (MBP), myc, hemagglutinin (HA), cystein, streptavidin, polyarginine, elastin (ELP)-based biopolymer, a galactose-binding domain, a calmodulin-binding domain, a chitin-binding domain, a cellulose-binding domain, a thioredoxine-binding domain, an intein binding domain, a S-peptide-binding domain, and DNA.

3. The polypeptide structure according to claim 1, wherein the antibody-binding region is selected from the group consisting of protein G, protein A, a protein A/G mixture, protein L, an antibody-binding peptide and an antibody-binding nucleotide, which can bind selectively to the Fc-domain of an antibody.

4. The polypeptide structure according to claim 1, wherein the signaling molecule is a cell permeation signaling peptide.

5. The polypeptide structure according to claim 4, wherein the cell permeation signaling peptide is selected from the group consisting of oligoarginine, a TAT-peptide (YGRKKRRQRRR), a drosophila-derived Antp peptide, a VP22 peptide, and mph-1-btm.

6. The polypeptide structure according to claim 1, wherein said nanoparticle is selected from the group consisting of a light-absorbing/scattering nanoparticle, a fluorescent nanoparticle and a magnetic nanoparticle.

7. The polypeptide structure according to claim 6, wherein the light-absorbing/scattering nanoparticle is Au or Ag nanoparticle.

8. The polypeptide structure according to claim 6, wherein the fluorescent nanoparticle is a nanoparticle of a material selected from the group consisting of CdSe, CdSe/ZnS, CdTe/CdS, CdTe/CdTe, ZnSe/ZnS, ZnTe/ZnSe, PbSe, PbS InAs, InP, InGaP, InGaP/ZnS and HgTe; or a nanoparticle in which an organic or inorganic fluorescent dye is dispersed in a material selected from the group consisting of silica, titanium and polymer.

9. The polypeptide structure according to claim 6, wherein, the magnetic nanoparticle is a nanoparticle of a material selected from the group consisting of Fe₂O₃, Fe₃O₄, FePt, Co and gadolinium (Gd).

10. The polypeptide structure according to claim 1, wherein the antibody can selectively target various organelles in the cytoplasm or nucleus.

11. The polypeptide structure according to claim 1, wherein the antibody has a therapeutic function.

12. A DNA encoding the polypeptide structure of claim 1.

13. A recombinant vector containing the DNA of claim 12.

14. A recombinant microorganism transformed with the recombinant vector of claim 13.

15. A method for preparing the polypeptide structure of claim 1, which comprises culturing a recombinant microorganism transformed with a recombinant vector containing DNA encoding said polypeptide structure.

16. A multifunctional complex, in which an antibody and a nanoparticle are bound to the polypeptide structure of claim 1.


SEQUENCE LISTING (7 sequences)

SEQ ID NO: 1 - DNA, 54 bp, Artificial Sequence
  signal peptide codon
ggcggcggcggcggccgtaaaaaacgtcgtcagcgtcgtcgtggctataaatgc

SEQ ID NO: 2 - DNA, 48 bp, Artificial Sequence
  primer
catatgcaccaccaccaccaccacaaaggcgaaacaactactgaagct

SEQ ID NO: 3 - DNA, 72 bp, Artificial Sequence
  primer
catatgcgtcgtcgtcgtcgtcgtcgtcgtcgtggcggcggcggcagcaaaggcgaaacaactactgaagct

SEQ ID NO: 4 - DNA, 30 bp, Artificial Sequence
  primer
catatgaaaggcgaaacaactactgaagct

SEQ ID NO: 5 - DNA, 88 bp, Artificial Sequence
  primer
ctcgagttagcatttatagccacgacgacgctgacgacgttttttacggccgccgccgccgccttcagttaccgtaaaggtcttagtc

SEQ ID NO: 6 - DNA, 31 bp, Artificial Sequence
  primer
ctcgagttcagttaccgtaaaggtcttagtc

SEQ ID NO: 7 - DNA, 33 bp, Artificial Sequence
  primer
ctcgagttattcagttaccgtaaaggtcttagt